(19) United States
(12) Patent Application Publication
(10) Pub. No.: US 2024/0101527 A1
(43) Pub. Date: Mar. 28, 2024

(54) QUINOLONE CARBOXYLIC DERIVATIVES

(71) Applicant: TAIGEN BIOTECHNOLOGY CO., LTD., Taipei City (TW)

(72) Inventors: Chu-Chung Lin, Taipei City (TW); Hung-Chuan Chen, Taipei City (TW); Chiayn Chiang, Taipei City (TW); Chih-Ming Chen, Santa Clara, CA (US)

(73) Assignee: TAIGEN BIOTECHNOLOGY CO., LTD., Taipei City (TW)

(21) Appl. No.: 17/754,703

(22) PCT Filed: Oct. 23, 2020
(86) PCT No.: PCT/US2020/056966

§ 371 Date: Apr. 8, 2022

Related U.S. Application Data
(60) Provisional application No. 62/924,763, filed on Oct. 23, 2019.

(51) Int. Cl. C07D 401/04; A61P 31/04

(52) U.S. Cl. CPC C07D 401/04; A61P 31/04

(57) ABSTRACT

A compound of Formula (I) below, or a pharmaceutically acceptable salt, stereoisomer, solvate, or prodrug thereof: in which R₁, R₂, R₃, R₅, R₆, and R₇ are defined as in the SUMMARY section. Further disclosed are a method of using the above-described compound, salt, stereoisomer, solvate, or prodrug for treating microbial infections and a pharmaceutical composition containing the same.

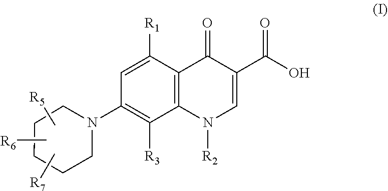

### TECHNICAL FIELD

This present disclosure is related to non-fluorinated quinolone carboxylic acid derivatives, pharmaceutical compositions containing the same and uses of the derivatives in the treatment of microbial infection.

### BACKGROUND

Infectious diseases have been the prime cause of death ever since human population existed, and microbial infection plays a serious threat to human life.

Antibiotics have been developed that counter microbial infection. However, antibiotic-resistant bacteria related infection has become the major challenge in the medical field for past decades.

Quinolones are well known antimicrobial agents which have been commercially available for more than 30 years. Quinolones include traditional fluoroquinolones (e.g., Gemifloxacin, Gatifloxacin, Moxifloxacin, and Levofloxacin) and non-fluorinated quinolones. Non-fluorinated quinolones (e.g., Nemonoxacin) differ from fluoroquinolones in that they lack the fluorine in the C6 position. See, e.g., Ledoussal Benoit et al., WO99/14214. The mode of action of the quinolones is through the inhibition of the bacterial DNA gyrase enzyme. Quinolones have been shown to be highly effective in the clinic, but wide-scale deployment of these current drugs jeopardizes their future long-term utility. As resistance to marketed antibiotics continues to increase, the new antibiotics would be expected to help address this unmet medical need.

### SUMMARY

The present disclosure is to provide the non-fluorinated quinolone carboxylic acid derivatives which have excellent activity and unexpected advantageous properties.

An aspect of this disclosure is drawn to the compounds of Formula (I) below, or pharmaceutically acceptable salts, stereoisomers, solvates, or prodrugs thereof:

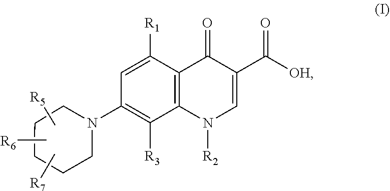

in this formula, R₁ is hydrogen; R₂ is cyclopropyl substituted with 1 to 3 halogens; R₃ is hydrogen, halogen, C₁₋₃ alkyl, or C₁₋₃ alkoxy, wherein each of C₁₋₃ alkyl and C₁₋₃ alkoxy is optionally substituted with 1 to 3 halogens; each of R₅, R₆, and R₇, independently, is hydrogen, halogen, C₁₋₃ alkyl or NH₂, wherein C₁₋₃ alkyl is optionally substituted with 1 to 3 halogens, provided that one of R₅, R₆, and R₇ is NH₂.

Also provided herein are pharmaceutical compositions comprising a compound disclosed herein, e.g., a compound of Formula (I), including a stereoisomer, an enantiomer, a pharmaceutically acceptable salt, a solvate, or a prodrug thereof, and one or more pharmaceutically acceptable carriers or excipients thereof. The pharmaceutical composition can be used for treating microbial infections or diseases associated with the pathogenic microorganisms.

Further provided herein is a method of treating, preventing, or ameliorating the microbial infections, or one or more symptoms of a pathogenic microorganism-mediated disorder, disease, or condition in a subject in need thereof, comprising administering to the subject a therapeutically effective amount of a compound disclosed herein, e.g., a compound of Formula (I), including a stereoisomer, an enantiomer, a pharmaceutically acceptable salt, a solvate, or a prodrug thereof. In related embodiments, the method may further comprise administering a compound disclosed herein, e.g., a compound of Formula (I), including a stereoisomer, an enantiomer, a pharmaceutically acceptable salt, a solvate, or a prodrug thereof, in combination with one or more additional therapeutic agents, wherein a compound disclosed herein and one or more additional therapeutic agents are administered either together in a single formulation, or administered separately in different formulations, and wherein the administration of the compound disclosed herein and the additional therapeutic agents is done concomitantly, or in series.

Additionally provided herein is a method of preparing a compound disclosed herein, e.g., a compound of Formula (I), including a stereoisomer, an enantiomer variant, a pharmaceutically acceptable salt, a solvate, or a prodrug thereof.

### DETAILED DESCRIPTION

To facilitate understanding of the disclosure set forth herein, a number of terms are defined below.

Generally, the nomenclature used herein and the laboratory procedures in organic chemistry, medicinal chemistry, and pharmacology described herein are those well-known and commonly employed in the art. Unless defined otherwise, all technical and scientific terms used herein generally have the same meaning as commonly understood by one of ordinary skill in the art to which this disclosure belongs.

The term “about” will be understood by persons of ordinary skill in the art and will vary to some extent on the context in which it is used. As used herein, when referring to a measurable value such as an amount, a temporal duration, and the like, the term “about” is meant to encompass variations of ±20% or ±10%, including ±5%, ±1%, and +0.1% from the specified value, as such variations are appropriate to perform the disclosed methods.

The terms “treat,” “treating,” and “treatment” are meant to include alleviating or abrogating a disorder, disease, or condition, or one or more of the symptoms associated with the disorder, disease, or condition; or alleviating or eradicating the cause(s) of the disorder, disease, or condition itself.

The terms “prevent,” “preventing,” and “prevention” are meant to include a method of delaying and/or precluding the onset of a disorder, disease, or condition, and/or its attendant symptoms; barring a subject from acquiring a disorder, disease, or condition; or reducing a subject's risk of acquiring a disorder, disease, or condition.

The terms “patient,” “individual” or “subject” refer to a human or a non-human mammal. In one embodiment, the patient, individual, or subject is human.

The term “therapeutically effective amount” refers to the amount of an active compound that is sufficient to prevent development of, or alleviate to some extent, one or more of the symptoms of the disorder, disease, or condition being treated.

The term “pharmaceutically acceptable carrier” or “pharmaceutically acceptable excipient” refers to a pharmaceutically acceptable material, composition, or vehicle, such as a liquid or solid filler, diluents, solvent, or encapsulating material, which does not abrogate the biological activity or properties of the compound, and is relatively non-toxic, i.e., the material may be administered to an individual without causing an undesirable biological effect or interacting in a deleterious manner with any of the components of the composition in which it is contained. See, Remington: The Science and Practice of Pharmacy, 21st ed.; Lippincott Williams & Wilkins: Philadelphia, PA, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, F L, 2009.

The term “one or more” refers to either one or a number above one (e.g., 2, 3, 4, 5, 6, 7 or above).

The term “halo” or “halogen” (alone or as part of another substituent) refers to a fluorine, chlorine, bromine, or iodine atom.

The term “C1-3 alkyl” (alone or in combination with another term) refers to a straight- or branched-chain saturated hydrocarbyl substituent containing 1 to 3 carbon atoms. Examples of C₁₋₃ alkyl include methyl, ethyl, n-propyl, isopropyl, and the like.

The term “C₁₋₃ alkoxy” (alone or in combination with another term) refers to the group —OR′ wherein R′ is C₁₋₃ alkyl. Examples of C₁₋₃ alkoxy include methoxy, ethoxy, n-propoxy, and isopropoxy.

The term “hydroxyl protecting group” refers to a chemical group that blocks the OH function for further reactions and can be removed under controlled condition. The hydroxyl protecting groups are well known in the art, representative protecting groups include, but are not limited to, allyl (All), methoxymethyl (MOM), 2-methoxyethoxymethyl (MEM), methylthiomethyl (MTM), benzyloxymethyl (BOM), 2-(trimethylsilyl)ethoxymethyl (SEM), tetrahydropyranyl (THP), 2,4-dinitrobenzyl, diphenylmethyl (DPM), trityl (Tr), p-methoxyphenyldiphenylmethyl (MMTr), benzyl (Bn), naphthyl (NAP), p-methoxybenzyl (PMB), p-nitrobenzyl, formyl, acyl (Ac), chloroacyl, methoxyacyl, pivaloyl (Piv), benzoyl (Bz), p-nitrobenzoyl, p-methoxybenzoyl, p-bromobenzoyl, p-phenylbenzoyl, trimethylsilyl (TMS), tnethylsilyl (TES), isopropyldimethylsilyl (IPDMS), triisopropylsilyl (TIPS), tert-butyldimethylsilyl (TBS), tert-butyldiphenylsilyl (TBDPS), methyldiphenylsilyl, thexyldimethylsilyl (TDS), methyl carbonate, ethyl carbonate, 2,2,2-trichloroethyl carbonate (Troc), allyl carbonate (Alloc), 9-(Fluorenylmethyl) carbonate (Fmoc), benzyl carbonate (Cbz), t-butyl carbonate (Boc), sulfate, allylsulfonate, methanesulfonate, benzylsulfonate, and tosylate.

The term “solvate” refers to a complex formed between an active compound and a pharmaceutically acceptable solvent. Examples of pharmaceutically acceptable solvents include water, ethanol, isopropanol, ethyl acetate, acetic acid, and ethanolamine.

The disclosure provided herein relates to a compound of Formula (I), or a pharmaceutically acceptable salt, stereoisomer, solvate, or prodrug thereof:

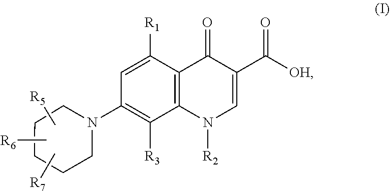

wherein
R₁ is hydrogen;
R₂ is cyclopropyl substituted with 1 to 3 halogens;
R₃ is hydrogen, halogen, C₁₋₃ alkyl, or C₁₋₃ alkoxy, wherein each of C₁₋₃ alkyl and C₁₋₃ alkoxy is optionally substituted with 1 to 3 halogens;
each of R₅, R₆, and R₇, independently, is hydrogen, halogen, C₁₋₃ alkyl or NH₂, wherein C₁₋₃ alkyl is optionally substituted with 1 to 3 halogens, provided that only one of R₅, R₆, and R₇ is NH₂.

In one embodiment, provided herein is a compound of Formula (II), or a pharmaceutically acceptable salt, stereoisomer solvate, or prodrug thereof:

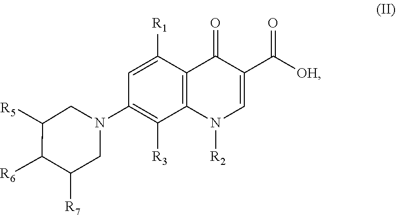

wherein
R₁ is hydrogen;
R₂ is cyclopropyl substituted with 1 to 3 halogens;
R₃ is hydrogen, halogen, C₁₋₃ alkyl, or C₁₋₃ alkoxy, wherein each of C₁₋₃ alkyl and C₁₋₃ alkoxy is optionally substituted with 1 to 3 halogens;
each of R₅, R₆, and R₇, independently, is hydrogen, halogen, C₁₋₃ alkyl or NH₂, wherein C₁₋₃ alkyl is optionally substituted with 1 to 3 halogens, provided that only one of R₅, R₆, and R₇ is NH₂.

In another embodiment, provided herein is a compound of Formula (III), or a pharmaceutically acceptable salt, stereoisomer, solvate, or prodrug thereof:

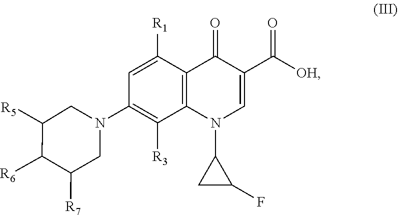

wherein
R₃ is hydrogen, halogen, C₁₋₃ alkyl, or C₁₋₃ alkoxy, wherein each of C₁₋₃ alkyl and C₁₋₃ alkoxy is optionally substituted with 1 to 3 halogens;
each of R₅, R₆, and R₇, independently, is hydrogen, halogen, C₁₋₃ alkyl or NH₂, wherein C₁₋₃ alkyl is optionally substituted with 1 to 3 halogens, provided that only one of R₅, R₆, and R₇ is NH₂.

The following embodiments are inclusive of definitions for Formula (I), (II) and/or (III).

In one embodiment, R₂ is cyclopropyl substituted with one halogen. In another embodiment, R₂ is cyclopropyl substituted with one fluorine.

In one embodiment, R₃ is halogen, C₁₋₃ alkyl, or C₁₋₃ alkoxy. In another embodiment, R₃ is chlorine, methyl, or methoxy.

In one embodiment, R₅ is hydrogen, halogen, C₁₋₃ alkyl optionally substituted with 1 to 3 halogens, or NH₂. In another embodiment, R₅ is hydrogen, fluorine, C₁₋₃ alkyl, or NH₂. In yet another embodiment, R₅ is hydrogen or NH₂. In other embodiment, R₅ is hydrogen. In other embodiment, R₅ is NH₂.

In one embodiment, R₆ is hydrogen, halogen, C₁₋₃ alkyl optionally substituted with 1 to 3 halogens, or NH₂. In another embodiment, R₆ is hydrogen, fluorine, C₁₋₃ alkyl optionally substituted with 1 to 3 fluorines, or NH₂. In yet another embodiment, R₆ is NH₂. In other embodiment, R₆ is hydrogen, fluorine, or C₁₋₃ alkyl optionally substituted with 1 to 3 fluorines.

In one embodiment, R₇ is hydrogen, halogen, or C₁₋₃ alkyl optionally substituted with 1 to 3 halogens. In another embodiment, R₇ is hydrogen, halogen, or C₁₋₃ alkyl. In yet another embodiment, R₇ is hydrogen, fluorine, or methyl.

In one embodiment, the compound provided herein is selected from the group consisting of:

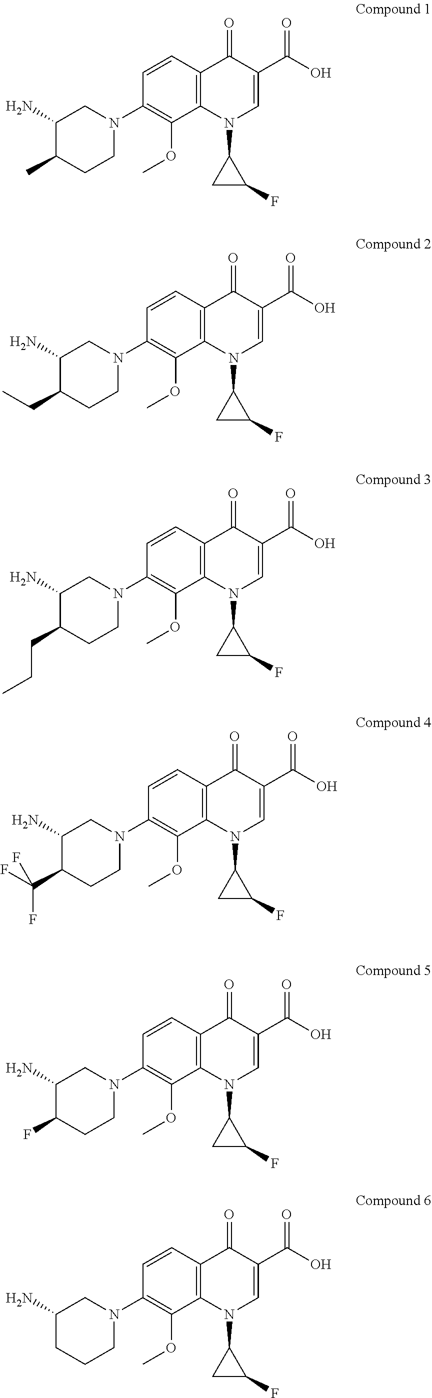 [SMILES: COc1c(N2CC[C@@H](C)[C@H](N)C2)ccc2c(=O)c(C(=O)O)cn([C@@H]3C[C@@H]3F)c12 ; CC[C@@H]1CCN(c2ccc3c(=O)c(C(=O)O)cn([C@@H]4C[C@@H]4F)c3c2OC)C[C@H]1N ; CCC[C@@H]1CCN(c2ccc3c(=O)c(C(=O)O)cn([C@@H]4C[C@@H]4F)c3c2OC)C[C@H]1N ; COc1c(N2CC[C@@H](C(F)(F)F)[C@H](N)C2)ccc2c(=O)c(C(=O)O)cn([C@@H]3C[C@@H]3F)c12 ; COc1c(N2CC[C@@H](F)[C@H](N)C2)ccc2c(=O)c(C(=O)O)cn([C@@H]3C[C@@H]3F)c12 ; COc1c(N2CCC[C@H](N)C2)ccc2c(=O)c(C(=O)O)cn([C@@H]3C[C@@H]3F)c12]

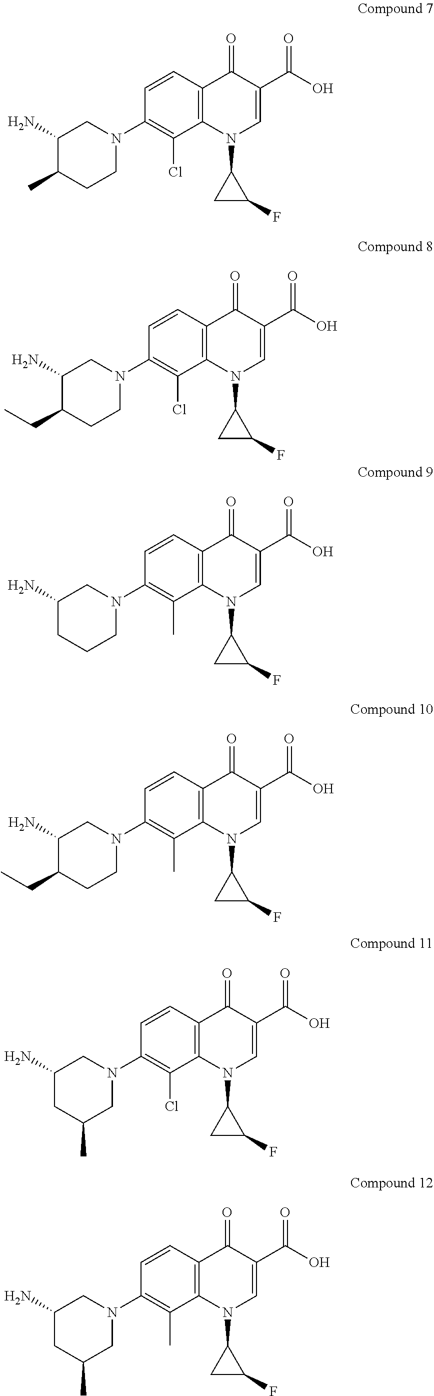 [SMILES: C[C@@H]1CCN(c2ccc3c(=O)c(C(=O)O)cn([C@@H]4C[C@@H]4F)c3c2Cl)C[C@H]1N ; CC[C@@H]1CCN(c2ccc3c(=O)c(C(=O)O)cn([C@@H]4C[C@@H]4F)c3c2Cl)C[C@H]1N ; Cc1c(N2CCC[C@H](N)C2)ccc2c(=O)c(C(=O)O)cn([C@@H]3C[C@@H]3F)c12 ; CC[C@@H]1CCN(c2ccc3c(=O)c(C(=O)O)cn([C@@H]4C[C@@H]4F)c3c2C)C[C@H]1N ; C[C@H]1C[C@H](N)CN(c2ccc3c(=O)c(C(=O)O)cn([C@@H]4C[C@@H]4F)c3c2Cl)C1 ; Cc1c(N2C[C@@H](C)C[C@H](N)C2)ccc2c(=O)c(C(=O)O)cn([C@@H]3C[C@@H]3F)c12]

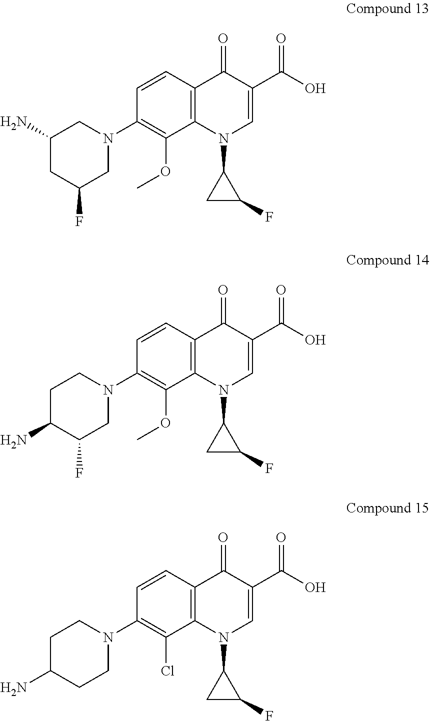 [SMILES: COc1c(N2C[C@@H](N)C[C@H](F)C2)ccc2c(=O)c(C(=O)O)cn([C@@H]3C[C@@H]3F)c12 ; COc1c(N2CC[C@H](N)[C@@H](F)C2)ccc2c(=O)c(C(=O)O)cn([C@@H]3C[C@@H]3F)c12 ; NC1CCN(c2ccc3c(=O)c(C(=O)O)cn([C@@H]4C[C@@H]4F)c3c2Cl)CC1]

It has been found that the compounds of this disclosure are effective antimicrobial agents against a broad range of pathogenic microorganisms with advantages in unexpected antimicrobial activity and low susceptibility to microbial resistance.

The compounds provided herein are intended to encompass all possible stereoisomers, unless a particular stereochemistry is specified. Where the compound provided herein contains an alkenyl or alkenylene group, the compound may exist as one or a mixture of geometric cis/trans (or Z/E) isomers. Where structural isomers are interconvertible, the compound may exist as a single tautomer or a mixture of tautomers. This can take the form of proton tautomerism in the compound that contains, for example, an imino, keto, or oxime group; or so-called valence tautomerism in the compound that contains an aromatic moiety. It follows that a single compound may exhibit more than one type of isomerism.

The compounds provided herein may be enantiomerically pure, such as a single enantiomer or a single diastereomer, or be stereoisomeric mixtures, such as a mixture of enantiomers, e.g., a racemic mixture of two enantiomers; or a mixture of two or more diastereomers. As such, one of ordinary skill in the art will recognize that administration of a compound in its (R) form is equivalent, for compounds that undergo epimerization in vivo, to administration of the compound in its (S) form. Conventional techniques for the preparation/isolation of individual enantiomers include synthesis from a suitable optically pure precursor, asymmetric synthesis from achiral starting materials, or resolution of an enantiomeric mixture, for example, chiral chromatography, recrystallization, resolution, diastereomeric salt formation, or derivatization into diastereomeric adducts followed by separation.

When the compound provided herein contains an acidic or basic moiety, it may also be provided as a pharmaceutically acceptable salt. The pharmaceutically acceptable salts are generally prepared from pharmaceutically acceptable non-toxic bases or acids including inorganic or organic bases and inorganic or organic acids. Suitable acids for use in the preparation of pharmaceutically acceptable salts include, but are not limited to, acetate, ascorbate, adipate, alginate, aspirate, benzenesulfonate, benzoate, bicarbonate, bisulfate, bitartrate, borate, bromide, butyrate, camphorate, camphorsulfonate, camsylate, carbonate, chloride, clavulanate, citrate, cyclopentane propionate, diethylacetic, digluconate, dihydrochloride, dodecylsulfanate, edetate, edisylate, estolate, esylate, ethanesulfonate, formic, fumarate, gluceptate, glucoheptanoate, gluconate, glutamate, glycerophosphate, glycollylarsanilate, hemisulfate, heptanoate, hexanoate, hexylresorcinate, hydrabamine, hydrobromide, hydrochloride, 2-hydroxyethanesulfonate, hydroxynaphthoate, iodide, isonicotinic, isothionate, lactate, lactobionate, laurate, malate, maleate, mandelate, mesylate, methylbromide, methylnitrate, methylsulfate, methanesulfonate, mucate, 2-naphthalenesulfonate, napsylate, nicotinate, nitrate, N-methylglucamine ammonium salt, oleate, oxalate, pamoate (embonate), palmitate, pantothenate, pectinate, persulfate, phosphate/diphosphate, pimelic, phenylpropionic, polygalacturonate, propionate, salicylate, stearate, sulfate, subacetate, succinate, tannate, tartrate, teoclate, thiocyanate, tosylate, triethiodide, trifluoroacetate, undeconate, valerate and the like. Suitable bases for use in the preparation of pharmaceutically acceptable salts include, but are not limited to, magnesium hydroxide, calcium hydroxide, potassium hydroxide, zinc hydroxide, sodium hydroxide, primary, secondary, tertiary, and quaternary, aliphatic and aromatic amines, including L-arginine, benethamine, benzathine, choline, deanol, diethanolamine, diethylamine, dimethylamine, dipropylamine, diisopropylamine, 2-(diethylamino)-ethanol, ethanolamine, ethylamine, ethylenediamine, isopropylamine, N-methyl-glucamine, hydrabamine, 1H-imidazole, L-lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, secondary amines, triethanolamine, trimethylamine, triethylamine, N-methyl-D-glucamine, 2-amino-2-(hydroxymethyl)-1,3-propanediol, tromethamine and the like.

The compound provided herein may also be provided as a prodrug, which is a functional derivative of the compound, for example, of Formula (I), and is readily convertible into the parent compound in vivo. Prodrugs are often useful because, in some situations, they may be easier to administer than the parent compound. They may, for instance, be bioavailable by oral administration whereas the parent compound is not. The prodrug may also have enhanced solubility in pharmaceutical compositions over the parent compound. A prodrug may be converted into the parent drug by various mechanisms, including enzymatic processes and metabolic hydrolysis.

Provided herein are pharmaceutical compositions comprising a compound provided herein, e.g., a compound of Formula (I), as an active ingredient, including a stereoisomer, a diastereomer, a pharmaceutically acceptable salt, a solvate, or a prodrug thereof; and one or more pharmaceutically acceptable carriers or excipients.

Suitable carriers or excipients are well known to those skilled in the art, and non-limiting examples of suitable excipients are provided herein. Whether a particular excipient is suitable for incorporation into a pharmaceutical composition or dosage form depends on a variety of factors well known in the art, including, but not limited to, the method of administration. The carriers or excipients include binders, fillers, diluents, disintegrants, pH adjusters, wetting agents, lubricants, glidants, coloring agents, dye-migration inhibitors, sweetening agents, flavoring agents, emulsifying agents, suspending and dispersing agents, preservatives, solvents, non-aqueous liquids, organic acids, and sources of carbon dioxide. Examples of the carriers or excipients include, but are not limited to, water, lactose, dextrose, fructose, sucrose, sorbitol, mannitol, polyethylene glycol, propylene glycol, starch, gum, gelatin, alginate, calcium silicate, calcium phosphate, cellulose, aqueous syrup, methylcellulose, polyvinylpyrrolidone, alkyl parahydroxybenzosorbate, talc, magnesium stearate, stearic acid, glycerin, various oils such as sesame oil, olive oil, and soybean oil, and the like.

The pharmaceutical compositions of the present disclosure comprise a compound provided herein (e.g., a compound of Formula (I), including a stereoisomer, a diastereomer, a pharmaceutically acceptable salt, a solvate, or a prodrug thereof) as an active ingredient, one or more pharmaceutically acceptable carriers/excipients and optionally other therapeutic ingredients or adjuvants. The compositions include compositions suitable for oral, rectal, topical, and parenteral (including subcutaneous, intramuscular, and intravenous) administration. These dosage forms can be prepared according to conventional methods and techniques known to those skilled in the art.

The pharmaceutical compositions provided herein can be provided in a unit-dosage form or multiple-dosage form. A unit-dosage form, as used herein, refers to physically discrete a unit suitable for administration to a human and animal subject, and packaged individually as is known in the art. Each unit-dose contains a predetermined quantity of an active ingredient(s) sufficient to produce the desired therapeutic effect, in association with the required pharmaceutical carriers or excipients. Examples of a unit-dosage form include an ampoule, syringe, and individually packaged tablet and capsule. For example, a 100 mg unit dose contains about 100 mg of an active ingredient in a packaged tablet or capsule. A unit-dosage form may be administered in fractions or multiples thereof. A multiple-dosage form is a plurality of identical unit-dosage forms packaged in a single container to be administered in segregated unit-dosage form. Examples of a multiple-dosage form include a vial, bottle of tablets or capsules, or bottle of pints or gallons.

The pharmaceutical compositions provided herein can be administered at once, or multiple times at intervals of time. It is understood that the precise dosage and duration of treatment may vary with the age, weight, and condition of the patient being treated, and may be determined empirically using known testing protocols or by extrapolation from in vivo or in vitro test or diagnostic data. It is further understood that for any particular individual, specific dosage regimens should be adjusted over time according to the individual need and the professional judgment of the person administering or supervising the administration of the formulations.

The pharmaceutical compositions provided herein for oral administration can be provided in solid, semisolid, or liquid dosage forms for oral administration. As used herein, oral administration also includes buccal, lingual, and sublingual administration. Suitable oral dosage forms include, but are not limited to, tablets, fastmelts, chewable tablets, capsules, pills, strips, troches, lozenges, pastilles, cachets, pellets, medicated chewing gum, bulk powders, effervescent or non-effervescent powders or granules, oral mists, solutions, emulsions, suspensions, wafers, sprinkles, elixirs, syrups, liposomes, micelles, microspheres, nanosystems, sustained release formulations, and the like.

The pharmaceutical compositions also provided herein can be administered parenterally by injection, infusion, or implantation, for local or systemic administration. Parenteral administration, as used herein, includes intravenous, intraarterial, intraperitoneal, intrathecal, intraventricular, intraurethral, intrasternal, intracranial, intramuscular, intrasynovial, intravesical, and subcutaneous administration. The pharmaceutical compositions provided herein for parenteral administration can be formulated in any dosage forms that are suitable for parenteral administration, including solutions, suspensions, emulsions, micelles, liposomes, microspheres, nanosystems, and solid forms suitable for solutions or suspensions in liquid prior to injection. Such dosage forms can be prepared according to conventional methods known to those skilled in the art of pharmaceutical science.

The pharmaceutical compositions also provided herein can be administered topically to the skin, orifices, or mucosa. The topical administration, as used herein, includes (intra)dermal, conjunctival, intracorneal, intraocular, ophthalmic, auricular, transdermal, nasal, vaginal, urethral, respiratory, and rectal administration. The pharmaceutical compositions provided herein can be formulated in any dosage forms that are suitable for topical administration for local or systemic effect, including emulsions, solutions, suspensions, creams, gels, hydrogels, ointments, dusting powders, dressings, elixirs, lotions, suspensions, tinctures, pastes, foams, films, aerosols, irrigations, sprays, suppositories, bandages, and dermal patches. The topical formulation of the pharmaceutical compositions provided herein can also comprise liposomes, micelles, microspheres, nanosystems, and mixtures thereof.

Also provided herein is a combination of a compound disclosed herein, e.g., a compound of Formula (I), including a stereoisomer, an enantiomer or a pharmaceutically acceptable salt, a solvate, or a prodrug thereof, with one or more additional therapeutic agents (including, but not limited to, a second and different antimicrobial agent). In one embodiment, the compounds of this disclosure are also useful in combination with one or more additional therapeutic agents for simultaneous, separate or sequential administration.

Also provided herein is a kit comprising a compound disclosed herein, e.g., a compound of Formula (I), including a stereoisomer, an enantiomer, a pharmaceutically acceptable salt, a solvate, or a prodrug thereof. The kit may further comprise instructions for use, e.g., for use in treating the microbial infections. The instructions for use are generally written instructions, although electronic storage media (e.g., magnetic diskette or optical disk) containing instructions are also acceptable.

Also provided herein is a method of treating, preventing, or ameliorating the microbial infections, or one or more symptoms of a pathogenic microorganism-mediated disorder, disease, or condition in a subject, comprising administering to the subject a therapeutically effective amount of a compound disclosed herein, e.g., a compound of Formula (I), including a stereoisomer, an enantiomer, a pharmaceutically acceptable salt, a solvate, or a prodrug thereof. Such microbial infections, or one or more symptoms of a pathogenic microorganism-mediated disorder include (for example) central nervous system infections, external ear infections, infections of the middle ear (such as acute otitis media), infections of the cranial sinuses, eye infections, infections of the oral cavity (such as infections of the teeth, gums and mucosa), upper respiratory tract infections, lower respiratory tract infections, including pneumonia, genitourinary infections, gastrointestinal infections, gynecological infections, septicemia, sepsis, peritonitis, bone and joint infections, skin and skin structure infections, bacterial endocarditis, burns, antibacterial prophylaxis of surgery, and antibacterial prophylaxis in post-operative patients or in immunosuppressed patients (such as patients receiving cancer chemotherapy, or organ transplant patients).

Described below are the procedures used to synthesize the exemplary compounds.

All the reagents and solvents were purchased from commercial sources and used without further purification unless otherwise indication. All the reactions were carried out under dry nitrogen or argon atmosphere and monitored by thin layer chromatography (TLC) using Merck Silica Gel 60 F₂₅₄ glass-backed plate. Column chromatography was performed by Merck Silica Gel 60 (0.040-0.063 mm, 230-400 mesh). ¹H NMR and ¹³C NMR spectra were measured by Varian Mercury-300 and Varian Bruker AVIII-500 spectrometers, and the chemical shifts (δ) were reported in parts per million (ppm) relative to the resonance of the solvent peak. Multiplicities are reported with the following abbreviations: s (singlet), d (doublet), t (triplet), q (quartet), quin (quintet), m (multiplet), or br (broad). Low-resolution mass spectra were measured by HP Hewlett Packard 1100 series.

The following Scheme (I) is the versatile methodology for synthesizing the compounds of Formula (I):

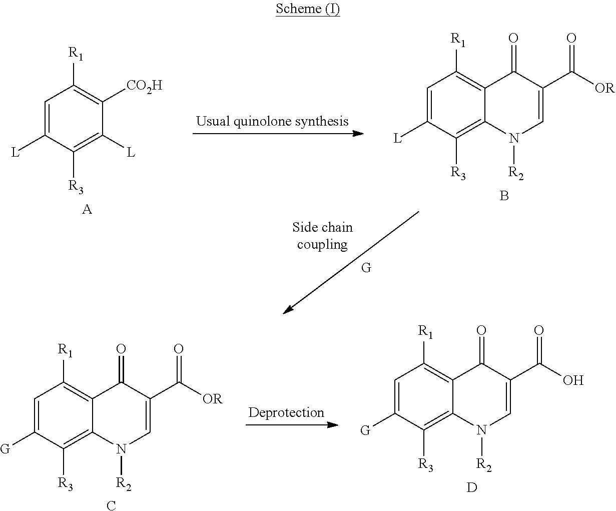

In one embodiment, a compound of Formula (I) can be prepared as shown in Scheme (I). Compound A is first converted to Compound B by the usual quinolone synthesis. Subsequently, Compound B is converted to Compound C by side chain coupling extension method. Compound C is deprotected to obtain Compound D. For the compounds shown in Scheme (I), R₁, R₂ and R₃ are defined as in any of the embodiments described herein. R is a hydroxyl protecting group.

The disclosure will be further understood by the following non-limiting examples.

### Example 1: Preparation and Characterization of Compounds 1 to 15

For all of the following examples, standard work-up and purification methods known to those skilled in the art can be utilized. Unless otherwise indicated, all temperatures are expressed in ° C. (degrees Centigrade). All reactions are conducted at room temperature (RT) unless otherwise noted. Synthetic methodologies used herein are intended to exemplify the applicable chemistry through the use of specific examples and are not indicative of the scope of the disclosure.

The starting materials used in the examples as described herein are either commercially available or can be prepared by a method known to one of ordinary skill in the art.

### Synthesis and Characterization of Compound 1:

### 7-(3-Amino-4-methyl-piperidin-1-yl)-1-(2-fluoro-cyclopropyl)-8-methoxy-4-oxo-1,4-dihydro-quinoline-3-carboxylic acid

Compound I-5 was first prepared from commercially available 2,4-difluoro-3-methoxy-benzoic acid via the route shown below:

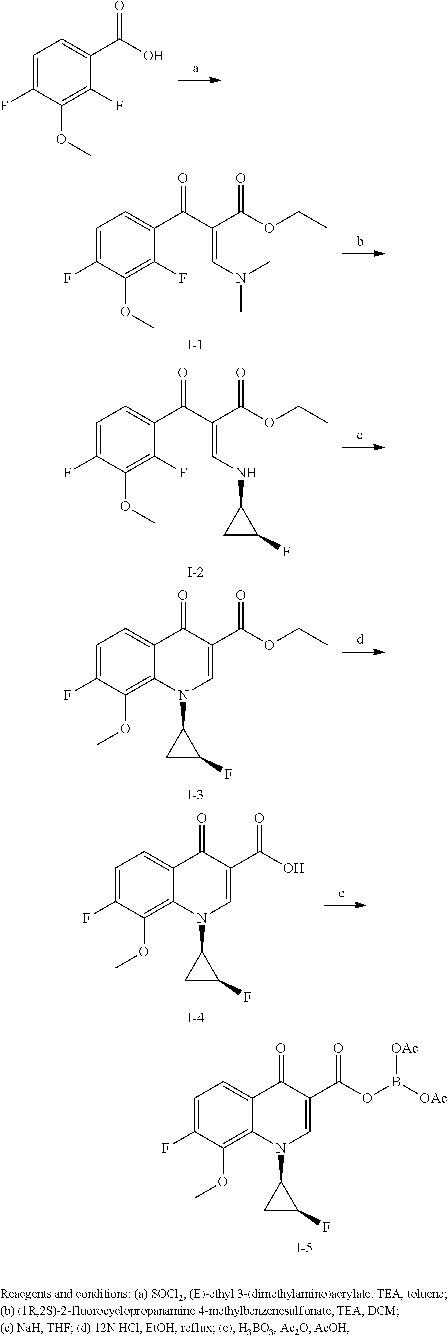

A solution of 2,4-difluoro-3-methoxy-benzoic acid (12 g, 63.8 mmol) in 100 mL of toluene at ambient temperature, dimethylformamide (DMF, 1 mL) and thionyl chloride (23 mL, 316 mmol) were added dropwise. The mixture was stirred at reflux and stirred for 3 h. The mixture was concentrated in vacuo to give a brown oil. To a stirred solution of (E)-ethyl 3-(dimethylamino)acrylate (11.8 mL, 107 mmol) and triethylamine (TEA, 13.8 mL, 95.6 mmol) in 50 mL of toluene was added dropwise a solution of the brown oil in 20 mL of toluene. The mixture was refluxed for 16 h. The mixture was washed with 100 mL of water, dried over MgSO₄ and concentrated in vacuo to give I-1 (20 g).

To a stirred solution of I-1 (20 g) in 300 mL of dichloromethane (DCM) in an ice bath was added TEA (20 mL, 138 mmol) and (1R,2S)-2-fluorocyclopropanamine-4-methylbenzenesulfonate (20 g, 80.8 mmol) and continuously stirred for 2 h. The mixture was washed with 200 mL of water, dried over MgSO₄ and concentrated in vacuo to give I-2 (21 g).

To a stirred solution of sodium hydride (2.7 g, 67.5 mmol) in 100 mL of toluene was added dropwise a solution of the I-2 (21 g) in 100 mL of toluene at 0° C. and stirred for 1 h. The mixture was added dropwise 100 mL of 10% H₂SO₄ at 0° C. and then stirred for 30 min. The precipitate was collected by filtration and washed with water to give I-3 (15 g, yield: 73%).

A mixture of I-3 (15 g, 46.4 mmol) in 200 mL of EtOH and 12N HCl (19.5 mL, 230 mmol) was heated to reflux and stirred for 16 h. After cooling the mixture, the precipitate was collected by filtration and washed with water to give I-4 (12.5 g, yield: 92%).

To a solution of H₃BO₃ (1 g, 16.2 mmol) in Ac₂O (21 mL, 222 mmol) was heated 115° C. and stirred for 30 min. Two fractions of H₃BO₃ (3.2 g, 51.6 mmol) were stepwisely added into reaction in 1 h at 115° C. The mixture was added AcOH (30 mL) at 115° C. and stirred for 30 min. The mixture was further added I-4 (12.5 g, 42.3 mmol) at 115° C. and stirred for 16 h. 200 mL of water was added into the mixture dropwise at 0° C. and stirred for 10 min. The precipitate was collected by filtration and washed with water to give I-5 (15 g, yield: 86%).

Further, Compound 1 was prepared via the route shown below:

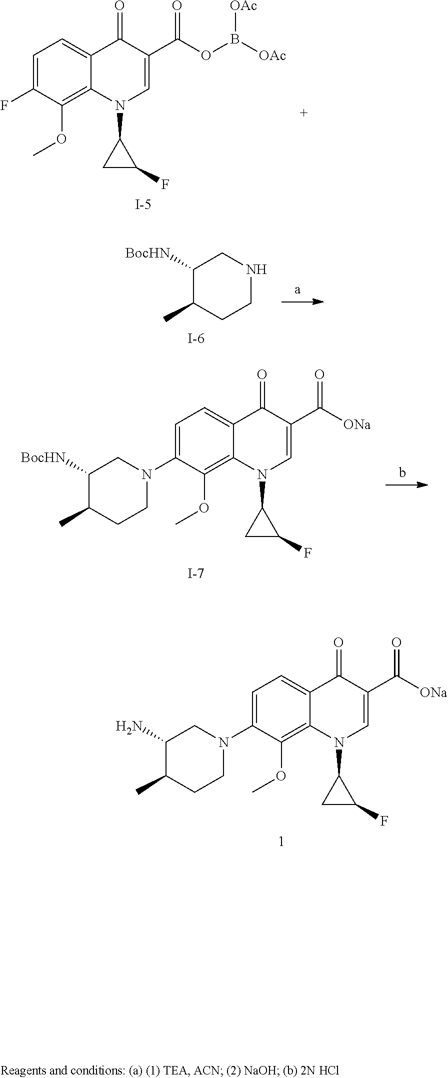

To a 10 mL flask, I-5 (0.17 g, 0.4 mmol), I-6 (0.10 g, 0.46 mmol) in acetonitrile (3 mL), triethylamine (0.08 mL, 0.6 mmol) was added. The reaction mixture was heated to 50° C. for 15 hours, and then the reaction mixture was cooled to 20° C., followed by addition of 10% NaOH (0.5 mL, 2.4 mmol) at 30±10° C., and stirred for one hour. The organic layer was collected after phase separation. The aqueous phase was extracted with 2 mL of acetonitrile and combined with the organic layer. The combined organic layers containing I-7 was used in the next step without further treatment.

To a stirred reaction of I-7 from the last step in 2 mL CH₂Cl₂, 0.33 mL 2N HCl was added. After being stirred for two hours at RT, the mixture was diluted with distilled water (2.5 mL). The aqueous solution was washed with dichloromethane (5 mL×3), followed by titration with 30% ammonia water to pH=7.8-8.0 to form a yellow powder slurry. The resulting slurry was cooled to 15±2° C. over a two-hour period and filtered by suction. The yellow powder was washed successively with distilled water (1 mL×2) and 95% EtOH (0.5 mL×2) and then dried on vacuum to give Compound 1 (0.1 g, yield: 67%). MS: m/z 390.2 (M+1); ¹H NMR (300 MHz, CD₃OD) δ ppm 8.83 (s, 1H), 8.14 (d, 1H), 7.35 (d, 1H), 5.06 (s, 1H), 4.18-4.08 (m, 2H), 3.83 (s, 1H), 3.14-3.09 (m, 1H), 2.92-2.85 (m, 2H), 1.99-1.94 (m, 1H), 1.74-1.70 (m, 4H), 1.19 (d, 3H).

Each of Compounds 2 to 15 was similarly prepared following the scheme as set forth above and the protocols described in the preparation of Compound 1.

Analytical data of Compounds 2 to 15 are listed below:

Compound 2: MS: m/z 404.2 (M+1); ¹H NMR (300 MHz, CD₃OD) δ ppm 8.84 (s, 1H), 8.15 (d, 1H), 7.36 (d, 1H), 5.08 (s, 1H), 4.19-4.03 (m, 2H), 3.85 (s, 1H), 3.31 (s, 1H), 2.97-2.90 (m, 2H), 2.14-1.34 (m, 7H), 1.06-1.01 (m, 3H).

Compound 3: MS: m/z 418.2 (M+1); ¹H NMR (300 MHz, CD₃OD) δ ppm 8.84 (s, 1H), 8.13 (s, 1H), 7.36 (s, 1H), 4.26-4.17 (m, 2H), 3.77-3.61 (m, 1H), 3.10-2.94 (m, 2H), 2.15-2.12 (m, 1H), 1.71-1.17 (m, 10H), 1.03-0.93 (m, 3H).

Compound 4: MS: m/z 444.2 (M+1); ¹H NMR (300 MHz, CD₃OD) δ ppm 8.87 (s, 1H), 8.18 (d, 1H), 7.39 (d, 1H), 5.07 (s, 1H), 4.16-3.72 (m, 4H), 3.29-2.71 (m, 3H), 2.29 (d, 1H), 1.98-1.67 (m, 3H).

Compound 5: MS: m/z 394.2 (M+1); ¹H NMR (300 MHz, CD₃OD) δ ppm 8.84 (s, 1H), 8.15 (d, 1H), 7.36 (d, 1H), 5.05-4.84 (m, 2H), 4.18-3.64 (m, 4H), 3.03 (t, 2H), 2.39-2.05 (m, 2H), 1.49 (m, 2H).

Compound 6: MS: m/z 380.2 (M+1); ¹H NMR (300 MHz, CD₃OD) δ ppm 8.85 (s, 1H), 8.16 (d, 1H), 7.34 (d, 1H), 4.17-4.15 (m, 1H), 3.92-3.85 (m, 2H), 3.70-3.51 (m, 2H), 3.29-2.94 (m, 2H), 2.22-1.47 (m, 6H).

Compound 7: MS: m/z 394.2 (M+1); ¹H NMR (300 MHz, CD₃OD) δ ppm 8.88 (s, 1H), 8.31 (d, 1H), 7.43 (d, 1H), 5.14 (s, 1H), 4.39 (s, 1H), 3.87-3.47 (m, 2H), 3.29-2.93 (m, 3H), 2.03-1.33 (m, 5H), 1.21 (d, 3H).

Compound 8: MS: m/z 408.2 (M+1); ¹H NMR (300 MHz, CD₃OD) δ ppm 8.89 (s, 1H), 8.35 (d, 1H), 7.45 (d, 1H), 5.15 (s, 1H), 4.38 (s, 1H), 3.85 (d, 1H), 3.53-2.99 (m, 4H), 2.17-2.14 (m, 1H), 1.98-1.32 (m, 6H), 1.07-1.02 (m, 3H).

Compound 9: MS: m/z 360.2 (M+1); ¹H NMR (300 MHz, CD₃OD) δ ppm 8.90 (s, 1H), 8.26 (d, 1H), 7.38 (d, 1H), 4.30-4.28 (m, 1H), 3.62-3.58 (m, 3H), 3.31-3.13 (m, 2H), 2.77 (s, 3H), 2.30-1.68 (m, 5H), 1.32-1.28 (m, 2H).

Compound 10: MS: m/z 388.2 (M+1); ¹H NMR (300 MHz, CD₃OD) δ ppm 8.87 (d, 1H), 8.25 (d, 1H), 7.36 (d, 1H), 4.27 (s, 1H), 3.68 (s, 1H), 3.38-2.91 (m, 5H), 2.17 (d, 1H), 1.84-1.23 (m, 9H), 1.07-1.02 (m, 3H).

Compound 11: MS: m/z 394.2 (M+1); ¹H NMR (300 MHz, CD₃OD) δ ppm 8.90 (d, 1H), 8.37 (d, 1H), 7.48 (d, 1H), 4.39-4.37 (m, 1H), 3.75 (s, 1H), 3.59-3.48 (m, 2H), 3.34-3.29 (m, 2H), 2.77 (t, 1H), 2.32 (br, 1H), 2.05-1.98 (m, 1H), 1.76-1.68 (m, 2H), 1.63-1.36 (m, 1H), 1.09-1.06 (m, 3H).

Compound 12: MS: m/z 374.2 (M+1); ¹H NMR (300 MHz, CD₃OD) δ ppm 8.86 (d, 1H), 8.26 (d, 1H), 7.34 (d, 1H), 4.27-4.23 (m, 1H), 3.77 (s, 1H), 3.38 (s, 1H), 2.79 (s, 3H), 2.62-2.59 (m, 1H), 2.32-1.70 (m, 6H), 1.33-1.07 (m, 5H).

Compound 13: MS: m/z 394.2 (M+1); ¹H NMR (300 MHz, CD₃OD) δ ppm 8.86 (s, 1H), 8.18 (d, 1H), 7.39 (d, 1H), 5.18-4.89 (m, 2H), 4.19-4.08 (m, 3H), 3.82-3.79 (m, 4H), 3.22-3.00 (m, 2H), 2.59-2.51 (m, 1H), 1.98-1.49 (m, 3H).

Compound 14: MS: m/z 394.2 (M+1); ¹H NMR (300 MHz, CD₃OD) δ ppm 8.89 (s, 1H), 8.16 (d, 1H), 7.38 (d, 1H), 5.08-4.73 (m, 1H), 4.26-4.13 (m, 3H), 3.85-3.81 (m, 4H), 3.53-3.49 (m, 1H), 3.30-2.99 (m, 2H), 2.26 (br, 1H), 1.98-1.58 (m, 3H).

Compound 15: MS: m/z 380.1 (M+1); ¹H NMR (300 MHz, CD₃OD) δ ppm 8.89 (s, 1H), 8.34 (d, 1H), 7.43 (d, 1H), 5.15 (s, 1H), 4.40-4.37 (m, 1H), 3.76-3.66 (m, 2H), 3.36-3.34 (m, 1H), 3.16-3.00 (m, 2H), 2.19-1.40 (m, 6H).

### Example 2: MIC (Minimum Inhibitory Concentration) Determination

The MICs of compounds against all bacterial strains were determined using the microdilution method according to the Clinical and Laboratory Standards Institute guidelines. Bacteria from two or three colonies were picked from freshly streaked culture plates and incubated in culture broth for 8 h. The bacterial culture was then diluted with double-concentrated culture broth to a final concentration of 5×10⁵ colony-forming unit (CFU)/mL. Testing compounds were prepared by diluting the stock solution with dimethyl sulfoxide (DMSO). The diluted bacterial suspension was added to an equal volume of a drug solution in a single microwell and incubated for 24 h at 37° C. The minimum concentration in the microwells with no visible bacterial growth was defined as the MIC. All MIC determinations were repeated twice on different days. The MIC of Compounds 1 to 15 against gram(+) and gram(−) bacteria are shown as follows:

| | MIC (μg/mL) | | | |
|---|---|---|---|---|
| | Gram(−) | | Gram(+) | |
| Compound | Pa27853* | PaK2376** | Sa29213*** | Sp49619**** |
| 1 | 0.25 | 1 | 0.031 | 0.031 |
| 2 | 0.5 | 2 | 0.031 | 0.031 |
| 3 | 2 | 4 | 0.031 | 0.031 |
| 4 | 4 | 4 | 0.031 | 0.031 |
| 5 | 4 | 16 | 0.063 | 0.063 |
| 6 | 2 | 8 | 0.063 | 0.063 |
| 7 | 2 | 2 | 0.031 | 0.031 |
| 8 | 0.125 | 0.5 | 0.031 | 0.008 |
| 9 | 0.5 | 2 | 0.031 | 0.031 |
| 10 | 0.125 | 0.5 | 0.031 | 0.031 |
| 11 | 2 | 4 | 0.031 | 0.25 |
| 12 | 1 | 4 | 0.063 | 0.125 |
| 13 | 2 | 16 | 0.063 | 0.031 |
| 14 | 2 | 2 | 0.063 | 0.125 |
| 15 | 0.25 | 8 | 0.063 | 0.063 |

*Pa27853: Pseudomonas aeruginosa ATCC 27853
**PaK2376: Pseudomonas aeruginosa K2376
***Sa29213: Staphylococcus aureus ATCC 29213
****Sp49619: Streptococcus pneumoniae ATCC 49619

Further, it was observed that compounds of this disclosure containing piperidine in position C7 and cyclopropyl substituted with fluorine in position C1 in Formula (I) unexpectedly exhibited higher potency in inhibiting the growth of bacteria than structurally close analogs. The results of the difference in MIC between the comparative compounds (structurally close analogs) and the example compound (containing piperidine in position C7 and cyclopropyl substituted with fluorine in position C1) are shown in the following table.

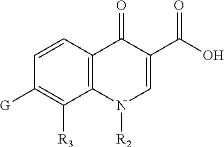

| | | | | | MIC difference (folds)* | | | |
|---|---|---|---|---|---|---|---|---|
| Comparative | | | | | Gram (−) | | Gram (+) | |
| compound | R₂ | R₃ | G | Compare with | Pa27853 | PaK2376 | Sa29213 | Sp49619 |
| A1 | 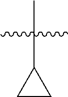 [SMILES: *C1CC1] | methoxy | 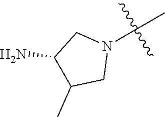 [SMILES: *N1CC(C)[C@H](N)C1] | Compound 1 | — | — | 16.1 | 64.5 |
| A2 | 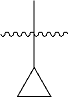 [SMILES: *C1CC1] | methoxy | 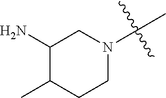 [SMILES: *N1CCC(C)C(N)C1] | Compound 1 | 32.0 | 32.0 | 4.0 | 4.0 |
| A3 | 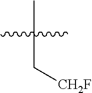 | methoxy | 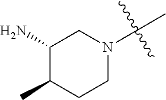 [SMILES: *N1CC[C@@H](C)[C@H](N)C1] | Compound 1 | 16.0 | 16.0 | 1.0 | 8.1 |
| B1 | 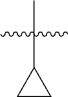 [SMILES: *C1CC1] | methoxy | 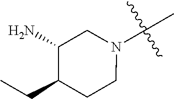 [SMILES: *N1CC[C@@H](CC)[C@H](N)C1] | Compound 2 | 2.0 | 2.0 | 8.1 | 1.0 |
| B2 | 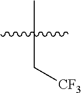 [SMILES: *CC(F)(F)F] | methoxy | 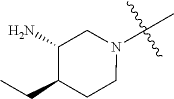 [SMILES: *N1CC[C@@H](CC)[C@H](N)C1] | Compound 2 | 16.0 | 4.0 | 1.0 | 4.0 |
| B3 | 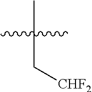 [SMILES: *CC(F)F] | methoxy | 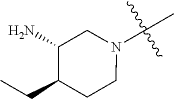 [SMILES: *N1CC[C@@H](CC)[C@H](N)C1] | Compound 2 | 4.0 | 8.0 | 1.0 | 8.1 |
| C1 | 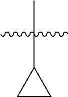 [SMILES: *C1CC1] | methoxy | 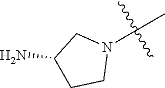 [SMILES: *N1CC[C@H](N)C1] | Compound 6 | — | — | 4.0 | 8.0 |
| C2 | 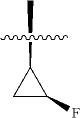 | methoxy | 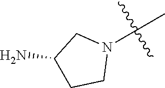 [SMILES: *N1CC[C@H](N)C1] | Compound 6 | 1.0 | 1.0 | 8.0 | 15.9 |
| C3 | 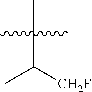 | methoxy | 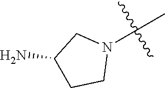 [SMILES: *N1CC[C@H](N)C1] | Compound 6 | 16.0 | 4.0 | 127.0 | 508.0 |
| D1 | 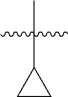 [SMILES: *C1CC1] | Cl | 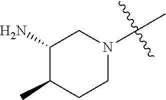 [SMILES: *N1CC[C@@H](C)[C@H](N)C1] | Compound 7 | 2.0 | 4.0 | 1.0 | 1.0 |
| E1 | 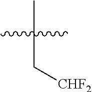 [SMILES: *CC(F)F] | Cl | 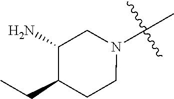 [SMILES: *N1CC[C@@H](CC)[C@H](N)C1] | Compound 8 | 8.0 | 8.0 | 1.0 | 15.6 |
| F1 | 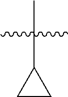 [SMILES: *C1CC1] | Cl | 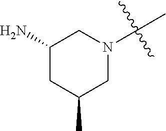 [SMILES: *N1C[C@@H](C)C[C@H](N)C1] | Compound 11 | 2.0 | 1.0 | 1.0 | 2.0 |
| F2 | 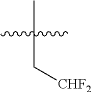 [SMILES: *CC(F)F] | Cl | 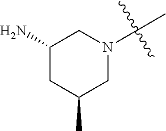 [SMILES: *N1C[C@@H](C)C[C@H](N)C1] | Compound 11 | 4.0 | 4.0 | 16.1 | 16.0 |

*MIC difference = (MIC value of a comparative compound)/(MIC value of an example compound)

These results indicate that the compounds of this disclosure unexpectedly exhibited higher potency in inhibiting the growth of bacteria, as compared to their structurally close analogs.

### Other Embodiments

All of the features disclosed in this specification may be combined in any combination. Each feature disclosed in this specification may be replaced by an alternative feature serving the same, equivalent or similar purpose. Thus, unless expressly stated otherwise, each feature disclosed is only an example of a series of equivalent or similar features.

From the above description, one skilled in the art can easily ascertain the essential characteristics of the present disclosure, and without departing from the scope thereof, can make various changes and modifications of the disclosure to adapt it to various usage and conditions. Thus, other embodiments are also within the scope of the following claims.

1. A compound of Formula (I), or a pharmaceutically acceptable salt, stereoisomer, solvate, or prodrug thereof, 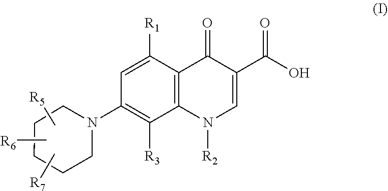
wherein
R₁ is hydrogen;
R₂ is cyclopropyl substituted with 1 to 3 halogens;
R₃ is hydrogen, halogen, C₁₋₃ alkyl, or C₁₋₃ alkoxy, wherein each of C₁₋₃ alkyl and C₁_3 alkoxy is optionally substituted with 1 to 3 halogens;
each of R₅, R₆, and R₇, independently, is hydrogen, halogen, C₁₋₃ alkyl or NH₂, wherein C₁₋₃ alkyl is optionally substituted with 1 to 3 halogens, provided that only one of R₅, R₆, and R₇ is NH₂.

2. The compound, or a pharmaceutically acceptable salt, stereoisomer, solvate, or prodrug thereof of claim 1, wherein the compound of Formula (I) is represented by Formula (II):

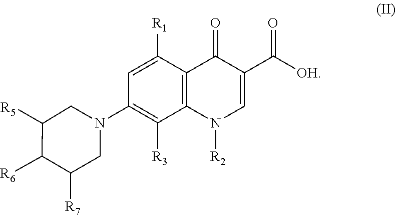

3. The compound, or a pharmaceutically acceptable salt, stereoisomer, solvate, or prodrug thereof of claim 1, wherein the compound of Formula (I) is represented by Formula (III):

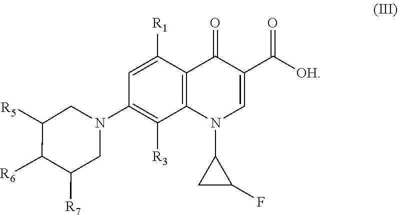

4. The compound, or a pharmaceutically acceptable salt, stereoisomer, solvate, or prodrug thereof of claim 1, wherein R₃ is chlorine, methyl, or methoxy.

5. The compound, or a pharmaceutically acceptable salt, stereoisomer, solvate, or prodrug thereof of claim 1, wherein R₅ is hydrogen or NH₂; R₆ is hydrogen, halogen, C₁₋₃ alkyl optionally substituted with 1 to 3 halogens, or NH₂; R₇ is hydrogen, halogen, or C₁₋₃ alkyl; provided that only one of R₅ and R₆ is NH₂.

6. The compound, or a pharmaceutically acceptable salt, stereoisomer, solvate, or prodrug thereof of claim 1, wherein R₃ is chlorine, methyl, or methoxy; R₅ is hydrogen or NH₂; R₆ is hydrogen, halogen, C₁₋₃ alkyl optionally substituted with 1 to 3 halogens, or NH₂; R₇ is hydrogen, halogen, or C₁₋₃ alkyl; provided that only one of R₅ and R₆ is NH₂.

7. The compound, or a pharmaceutically acceptable salt, stereoisomer, solvate, or prodrug thereof of claim 3, wherein R₃ is chlorine, methyl, or methoxy; R₅ is hydrogen or NH₂; R₆ is hydrogen, halogen, C₁₋₃ alkyl optionally substituted with 1 to 3 halogens, or NH₂; R₇ is hydrogen, halogen, or C₁₋₃ alkyl; provided that only one of R₅ and R₆ is NH₂.

8. The compound, or a pharmaceutically acceptable salt, stereoisomer, solvate, or prodrug thereof of claim 1, which is selected from the group consisting of:

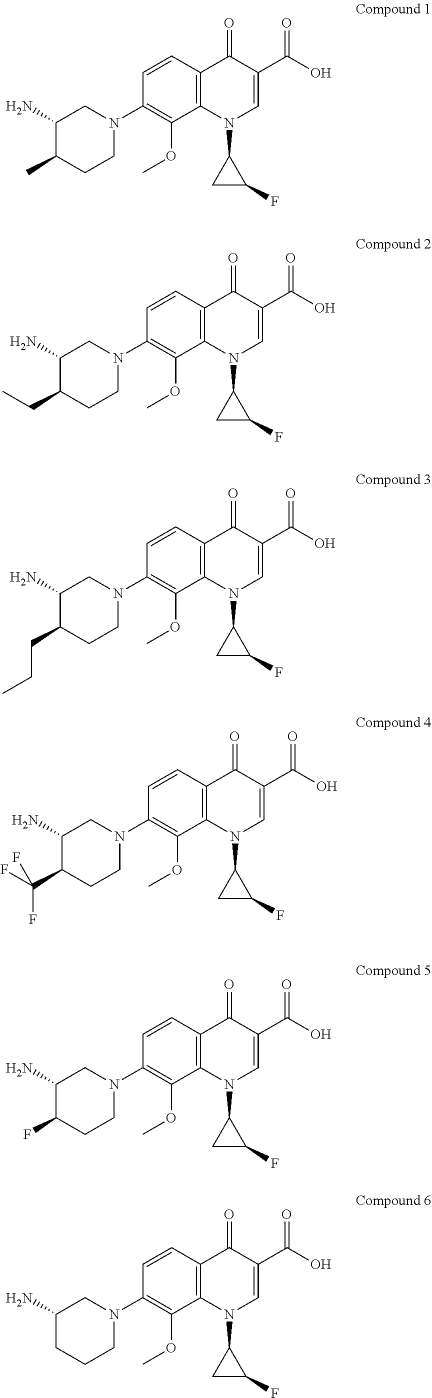 [SMILES: COc1c(N2CC[C@@H](C)[C@H](N)C2)ccc2c(=O)c(C(=O)O)cn([C@@H]3C[C@@H]3F)c12 ; CC[C@@H]1CCN(c2ccc3c(=O)c(C(=O)O)cn([C@@H]4C[C@@H]4F)c3c2OC)C[C@H]1N ; CCC[C@@H]1CCN(c2ccc3c(=O)c(C(=O)O)cn([C@@H]4C[C@@H]4F)c3c2OC)C[C@H]1N ; COc1c(N2CC[C@@H](C(F)(F)F)[C@H](N)C2)ccc2c(=O)c(C(=O)O)cn([C@@H]3C[C@@H]3F)c12 ; COc1c(N2CC[C@@H](F)[C@H](N)C2)ccc2c(=O)c(C(=O)O)cn([C@@H]3C[C@@H]3F)c12 ; COc1c(N2CCC[C@H](N)C2)ccc2c(=O)c(C(=O)O)cn([C@@H]3C[C@@H]3F)c12]

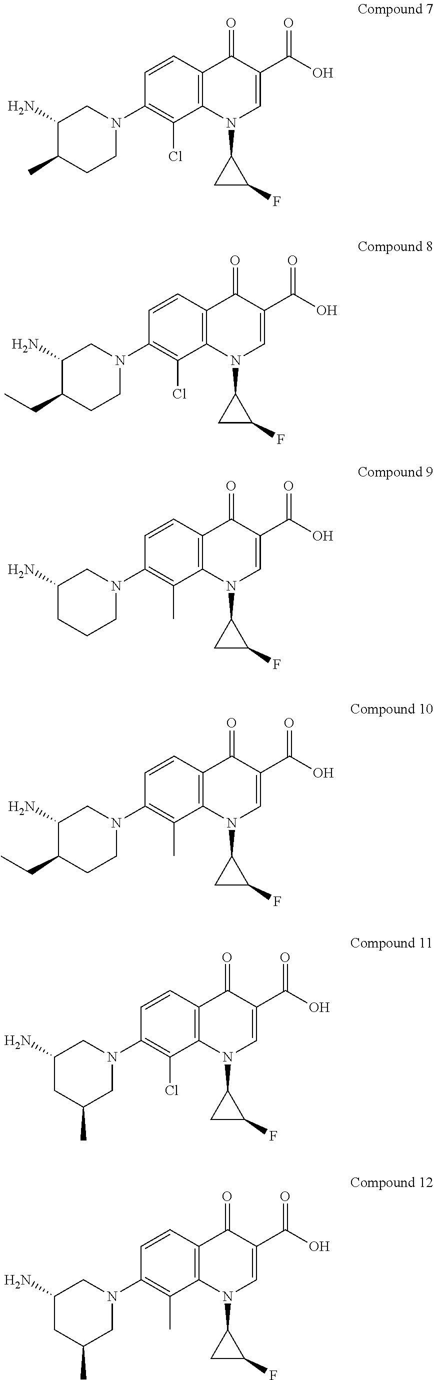 [SMILES: C[C@@H]1CCN(c2ccc3c(=O)c(C(=O)O)cn([C@@H]4C[C@@H]4F)c3c2Cl)C[C@H]1N ; CC[C@@H]1CCN(c2ccc3c(=O)c(C(=O)O)cn([C@@H]4C[C@@H]4F)c3c2Cl)C[C@H]1N ; Cc1c(N2CCC[C@H](N)C2)ccc2c(=O)c(C(=O)O)cn([C@@H]3C[C@@H]3F)c12 ; CC[C@@H]1CCN(c2ccc3c(=O)c(C(=O)O)cn([C@@H]4C[C@@H]4F)c3c2C)C[C@H]1N ; C[C@H]1C[C@H](N)CN(c2ccc3c(=O)c(C(=O)O)cn([C@@H]4C[C@@H]4F)c3c2Cl)C1 ; Cc1c(N2C[C@@H](C)C[C@H](N)C2)ccc2c(=O)c(C(=O)O)cn([C@@H]3C[C@@H]3F)c12]

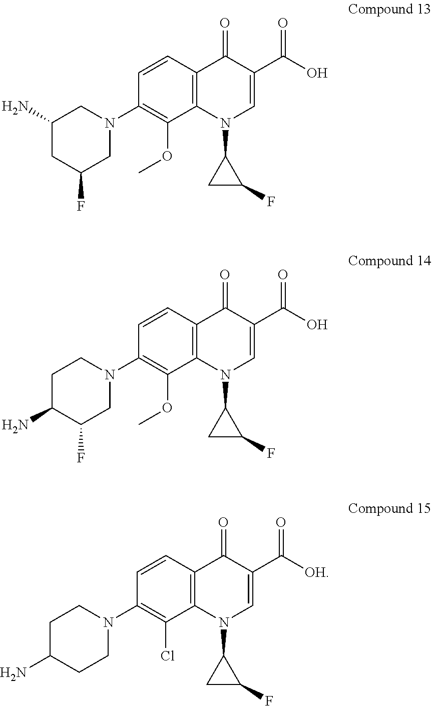 [SMILES: COc1c(N2C[C@@H](N)C[C@H](F)C2)ccc2c(=O)c(C(=O)O)cn([C@@H]3C[C@@H]3F)c12 ; COc1c(N2CC[C@H](N)[C@@H](F)C2)ccc2c(=O)c(C(=O)O)cn([C@@H]3C[C@@H]3F)c12 ; NC1CCN(c2ccc3c(=O)c(C(=O)O)cn([C@@H]4C[C@@H]4F)c3c2Cl)CC1]

9. A pharmaceutical composition comprising the compound, or the pharmaceutically acceptable salt, stereoisomer, solvate, or prodrug thereof of claim 1, and one or more pharmaceutically acceptable carriers thereof.

10. A compound, or a pharmaceutically acceptable salt, stereoisomer, solvate, or prodrug thereof of claim 1, for use in treatment, prevention, or amelioration of a microbial infection in a subject in need thereof.

11. A pharmaceutical composition of claim 9 for use in treatment, prevention, or amelioration of a microbial infection in a subject in need thereof.
